(19) United States
(12) Patent Application Publication
(10) Pub. No.: US 2014/0180133 A1
(43) Pub. Date: Jun. 26, 2014

(54) SCANNING ENDOSCOPIC IMAGING PROBES AND RELATED METHODS

(71) Applicants: Jeffrey Brennan, Los Angeles, CA (US); Mark S. Humayun, Glendale, CA (US)

(72) Inventors: Jeffrey Brennan, Los Angeles, CA (US); Mark S. Humayun, Glendale, CA (US)

(21) Appl. No.: 14/080,023

(22) Filed: Nov. 14, 2013

Related U.S. Application Data
(63) Continuation of application No. 13/479,798, filed on May 24, 2012.
Application No. 13/479,798 is a continuation of application No. 13/479,796, filed on May 24, 2012.
Application No. 13/479,796 is a continuation of application No. 13/905,707, filed on May 30, 2013.
Application No. 13/905,707 is a continuation of application No. 13/479,798, filed on May 24, 2012.
Application No. 13/479,798 is a continuation of application No. 13/479,796, filed on May 24, 2012.
Application No. 13/479,796 is a continuation of application No. 13/908,589, filed on Jun. 3, 2013.
Application No. 13/908,589 is a continuation of application No. 13/479,798, filed on May 24, 2012.
Application No. 13/479,798 is a continuation of application No. 13/479,796, filed on May 24, 2012.
(60) Provisional application No. 61/489,658, filed on May 24, 2011.
(60) Provisional application No. 61/489,658, filed on May 24, 2011.

(51) Int. Cl. A61B 5/00; A61M 5/178

(52) U.S. Cl. CPC A61B 5/0059; A61M 5/178
USPC 600/478

(57) ABSTRACT

An injection device with integrated imaging capability may include a hollow, tubular needle, an optical fiber disposed in parallel to the needle, a detector, a lens structure for focusing light coupled into the lens structure from the optical fiber at a focus beyond the distal end of the needle, and an imaging engine for processing a signal received from the detector via the optical fiber and the lens structure so as to generate an image of a region about the focus.

### CROSS-REFERENCE TO RELATED APPLICATION

This a continuation of U.S. patent application Ser. No. 13/479,798 (filed on May 24, 2012), Ser. No. 13/479,796 (filed on May 24, 2012), Ser. No. 13/905,707 (filed on May 30, 2013) and Ser. No. 13/908,589 (filed on Jun. 3, 2013), and also claims priority to and the benefit of U.S. Provisional Patent Application No. 61/489,658, filed on May 24, 2011. The foregoing applications are incorporated herein by reference in their entireties.

### TECHNICAL FIELD

In various embodiments, the present invention relates to endoscopic imaging probes, and, in particular, to handheld probes suitable for use in surgical procedures and for integration into surgical instruments.

### BACKGROUND

Advances in minimally invasive surgical procedures and the development of novel surgical instruments have enabled surgeons to access delicate areas of the body that were previously off-limits or only accessible through highly invasive procedures. These innovations have resulted in significant improvements in treatment options and patient outcomes for a variety of maladies. In addition, new diagnostic techniques—including new or improved imaging modalities—provide surgeons with more information and a better understanding of the area being treated, enabling them to collect, for example, real-time and non-destructive biopsies including analysis of regions that are typically difficult to access. One such useful diagnostic technique is optical coherence tomography (OCT), an interferometric technique for noninvasive diagnosis and imaging utilizing (typically infrared) light. OCT has transformed the field of ophthalmology and promises to have a similar impact on a variety of other medical specialties. OCT systems have become a mainstay in hospitals and ophthalmology clinics for diagnostic evaluation and imaging purposes. Furthermore, advances in technology have enabled smaller imaging device, such as, e.g., handheld endoscopic probes, that provide minimally invasive imaging of regions of interest not accessible using external imaging devices. Endoscopic probes are, during use, at least partially inserted into the patient's body. As will be appreciated by one of skill in the art, such probes impose particularly stringent requirements on size and maneuverability.

A particular mode of OCT, termed “A-scan,” provides one-dimensional axial depth scans of the tissue of interest, thus providing information on the identity, size, and depth of subsurface features. A series of spatially adjacent A-scans (typically lying in a straight line) may be combined to form a two-dimensional reconstructed image of the imaged area (termed a “B-scan”), and three-dimensional images, termed “C-scans,” may be formed by “stacking” multiple B-scans. B-scan formation typically requires the scanning of the optical beam across the surface of interest. For example, a surgeon may hold an OCT probe (from which the optical beam emanates) and move his or her hand to sweep the optical beam across the sample of interest. Alternatively, the probe may remain stationary while the beam direction is varied relative to the probe. In one configuration used for this purpose, the beam is deflected by 90° and the probe is rotated, causing a circular scan pattern in a plane perpendicular to the probe axis. Side-scanning in this manner is useful for imaging tubular organs, such as blood vessels or the esophagus. Another configuration, which facilitates forward-scanning, utilizes a pair of angle-cut rotating lenses that produce, in good approximation, a straight-line scan when rotating in opposite directions at the same angular speed. Alternative configurations for forward and/or side scanning utilize, e.g., a microelectromechanical-systems (MEMS) mirror to deflect the beam, or a piezo element to move the lens inside the probe.

In both side-scanning and forward-scanning probes, the lenses are optically coupled to a stationary external imaging console via optical fibers. In configurations that rely on lens rotation, two fibers, coupled by a rotary joint, are generally used to facilitate rotation of the lens relative to the console. Commercially available (“off-the-shelf”) rotary joints are, however, expensive. Moreover, their size precludes integration into the smaller, handheld probes (e.g., probes utilizing a 23-gauge needle) that are required, for example, for retinal surgery or similarly delicate procedures. Similarly, MEMS mirrors and piezo elements generally do not fit within a 23-gauge needle. Consequently, these components are typically mounted externally, placing constraints on the positioning and movement of the probe.

The positioning and orientation of the imaging probe is typically also subject to anatomical constraints. For example, retinal surgical procedures are typically performed via ports or cannulated incisions in the eye near the periphery of the cornea, as illustrated in FIG. 1. The imaging probe is, most naturally, inserted at an angle (between about 20° and about 60°) relative to the central region of the retina. Under this angle, neither the forward-scanning probe nor the side-scanning probe described above allows scanning the central region of the retina, which is often the area of greatest interest. In order to direct the beam perpendicularly at the central region using, e.g., a forward-scanning probe, the surgeon needs to contort the eye and place the probe in an unnatural position.

Further, as the number of tools to diagnose and treat the underlying condition expands, their combined utility is often curtailed by anatomical constraints. Retinal surgery, for example, generally relies on a variety of instruments (including, e.g., an illuminating light source, a treatment laser, a vitrector, an aspirator, etc.), which cannot all be introduced through the cannulated incisions into the eye simultaneously. Similarly, orthopedic procedures (e.g., knee reconstruction) typically involve a variety of instruments and tools, of which only a limited number can be inserted into the patient for access to the surgical site at any particular moment. The need to constantly swap out instruments because of limited access to the surgical site is frequently a problematic and time-consuming distraction to the surgeon.

In view of the various limitations of existing endoscopic imaging probes, there is a need for more compact (and, desirably, less costly) imaging probes that circumvent anatomical constraints, as well as for integrated devices that provide both imaging and treatment functionalities.

### SUMMARY

The present invention provides, in various embodiments, endoscopic imaging probes, and methods of operating them, that facilitate imaging an anatomic region of interest perpendicularly to the tissue surface while allowing the probe to be oriented at an angle to the surface. This flexibility is accomplished with an angle-cut lens, prism, or other structure that deflects the optical beam, typically by less than 90°, and focuses the light off-axis. Using a suitable actuation system, the lens may be rotated back and forth to scan the focus across the surface, resulting in a scan path following an arc segment. As long as the angle of rotation does not exceed a set threshold (e.g., 60°, or some other angle, depending on the particular application), the arc segment approximates a desired straight-line scan sufficiently for practical purposes.

In certain embodiments, the probe includes a single lens, which is connected to an exterior imaging console via an optical fiber. A rotary joint is not required since, in the intended mode of operation, the lens and fiber do not undergo full 360°-rotation, let alone multiple rotations in the same direction, but rotate by less than 180° (preferably by no more than 90°, and in certain embodiments by only 30° or less) in each direction. The simplified design of such a single-lens probe—compared, e.g., with that of a paired-angle-rotation scanning (PARS) probe as described above—renders it particularly suitable for small hand-held probes as well as, as a result of the reduced cost, for disposable probes. However, it is also possible to operate a dual-lens probe in a manner that achieves off-axis scanning and avoids the need for a rotary joint: by rotating only one of the lenses while keeping the second lens, which is coupled to the optical fiber, still.

For some applications, it is desirable to facilitate larger-angle rotations of the lens(es) and/or continuous rotation in the same direction, which generally requires a rotary joint. Various embodiments of the invention are directed to rotary joints that, due to greater compactness compared with that of off-the-shelf joints, can be integrated into the imaging probes, specifically, into tubular needles into which the lenses are assembled. In one embodiment, the fiber coupled to the lens and the fiber coupled to a fiber connector interfacing with the imaging console are aligned and held in place by a fiber ferrule and butt-coupled against each other. In another embodiment, the two fibers are coupled to each other via a pair of lenses that can rotate relative to one another. The small gap between the two fiber ends or the two coupling lenses, respectively, may be filled with an index-matching gel.

In addition to imaging probes with advantageous features, the present invention provides, in several embodiments, integrated probes having both imaging and treatment functionalities. One embodiment, for example, is directed to a vitrector, i.e., a surgical tool for extracting vitreous from the eye. (Consistently with its usage in the medical community, the term “vitreous” is, herein, used as a noun, denoting material from the vitreous body of the eye.) The vitrector includes an outer tube with a side window through which vitreous can enter, and an interior rotating or reciprocating cutter tube that provides the necessary shear forces for cutting the vitreous. An integrated imaging probe, including a lens mounted to the distal end of the cutter tube and moving along with the tube and an optical fiber run through the cutter tube, enables imaging the vitreous during the surgical procedure. Another embodiment is directed to an injection device that includes a fluid-delivery tube with integrated imaging components. Yet another embodiment provides a surgical drill device with a hollow core housing an optical fiber and lens. These hybrid devices facilitate monitoring the effect of the treatment procedure in real-time, and avoid the need to swap instruments. Further, in certain embodiments, they synergistically utilize the same actuation system to rotate or translate both the surgical tool and the imaging lens.

Accordingly, in a first aspect, the invention provides a scanning imaging probe including an optical fiber, and a lens assembly including a single lens structure (e.g., a gradient-index lens) mounted in a tube surrounding the fiber. The lens structure is placed at a distal end of the tube, optically coupled to the fiber, and shaped so as to deflect light coupled from the fiber into the lens structure and focus the light off-axis beyond the distal end. For example, the lens structure may consist of an angle-cut lens, or include or consist of a prism. The lens structure and the optical fiber may be aligned co-axially with each other. In some embodiments, the fiber is fused to the lens structure; in other embodiments, it is coupled to the lens structure via a fiber ferrule.

The imaging probe further includes an actuation mechanism for moving the lens assembly so as to scan the focus along a line. (As used herein, the term “line” is not limited to straight lines, but includes, e.g., arc segments or other curved lines. However, the term “linear” is, consistently with its usage in the technical field, used in reference to a straight-line scan.) In some embodiments, the actuation mechanism causes rotation of the lens assembly around an axis of the assembly (i.e., an axis of the tube). The rotation may be reciprocating and not exceed 90° (or, in some embodiments, 60° or 30°) in each direction. In some embodiments, the actuation mechanism causes reciprocation of the lens assembly along an axis of the assembly. The actuation mechanism may be a pneumatic, hydraulic, electromagnetic, or motor-driven mechanical actuation mechanism. In certain embodiments, the mechanism includes a transmission reconfigurable to dynamically alter the speed and/or the direction of actuation.

The outer diameter of the tube may less than 1 mm; in some embodiments, it is less than 520 μm. The tube may include or consist of a hypodermic needle, e.g., a 20-gauge needle, 23-gauge needle, a 25-gauge needle, or a 31-gauge needle. The probe may include an additional, outer tube surrounding the lens assembly, which remains stationary when the lens assembly moves. The probe may be a handheld probe, i.e., it may be sized for hand operation and, for example, include a handle having a shape and/or texture that facilitates a secure grip.

In a second aspect, the invention relates to a method of scanning tissue at a surface of the tissue, using an imaging probe that includes a rotatable lens structure shaped so as to focus a light beam exiting the lens structure off-axis beyond a distal end of the probe. The method involves positioning the probe such that the light beam, at a first rotational position of the lens structure, is incident on the tissue surface substantially perpendicularly, and scanning the tissue along an arc-shaped path by rotating the lens from a second rotational position to a third rotational position, wherein the second and third rotational positions are selected such that the lens structure passes through the first rotational position during the rotation. In some embodiments, the lens is rotated between the second and third positions in one rotational direction, and then back from the third to the second position in the opposite rotational direction. The rotation may be limited to (i.e., not exceed) a 90° angle, or, in some embodiments, a 30° angle. In certain embodiments, the method is practiced with an imaging probe that includes a second lens structure proximal to the rotatable lens structure, which couples light from an optical fiber to the rotatable lens structure; in this case, the method further includes keeping the second lens structure stationary while rotating the rotatable lens structure.

In a third aspect, a scanning imaging probe including two lens assemblies is provided. The first lens assembly includes a first tube and, mounted therein at a distal end, a first deflecting lens structure. The second lens assembly is coaxially disposed inside the first tube proximal to the first lens, and includes a second tube and, mounted therein at a distal end, a second deflecting lens structure. The second lens assembly is rotatable relative to the first tube. The probe includes actuation mechanisms associated with the first and second deflecting lens assemblies for rotating the lens assemblies independently of one another, The first and second lens assemblies are configured such that, when they are rotated, light is focused beyond the distal end of the first lens assembly and the focus is moved along a scan pattern. In some embodiments, the first and second deflecting lens structures are angle-cut lenses. The probe may further include an outer tube surrounding the first and second lens assemblies, the outer tube remaining stationary when the lens assemblies rotate.

The probe further includes a rotary joint disposed inside the second tube proximal to the second deflecting lens structure, and an optical fiber, coaxially disposed inside the second tube and optically coupling the rotary joint to the second deflecting lens structure, for coupling light into the second deflecting lens structure. In some embodiments, the rotary joint couples the optical fiber to a second optical fiber connectable to an imaging console, the second optical fiber remaining stationary when the second lens assembly rotates. The optical fiber and the second optical fiber may be axially aligned in a fiber ferrule and butt-coupled against each other (such that the coupling region and fiber ferrule collectively form the rotary joint). Alternatively, the two optical fibers may be coupled via a co-axial pair of collimating or converging lenses, which may be butt-coupled against each other. The gap between the two optical fibers or between the collimating lenses, respectively, may be filled with index-matching gel. In certain embodiments, the distance between the second deflecting lens structure and the rotary joint exceeds the coherence length of the light (which depends on the light source used).

A fourth aspect of the invention relates to a vitrector with integrated imaging capability. The vitrector includes a vitrector tube with a side port at a distal end through which vitreous can be admitted, and a suction mechanism associated with the vitrector tube, which draws the vitreous towards the proximal end of the vitrector tube. Further, the vitrector includes a tubular cutter, coaxially disposed in the vitrector tube, and, associated with the cutter, an actuation mechanism for moving the cutter relative to the vitrector tube so as to cut the vitreous for suctioning by the suction mechanism. The vitrector also includes an optical fiber coaxially disposed in the cutter, and a lens structure, disposed at a distal end of the tubular cutter and optically coupled to the optical fiber, for focusing light coupled into the lens structure from the optical fiber beyond the distal end so as to image a region about the focus.

The lens structure may be shaped to deflect the light and focus it off-axis (e.g., it may include or consist of an angle-cut lens), such that movement of the cutter simultaneously causes the focus to be scanned along a line. In some embodiments, the actuation mechanism is a rotary mechanism, causing the focus to be scanned along an arc segment. In other embodiments, the actuation mechanism is a reciprocating mechanism, causing the focus to be scanned along an axis parallel to an axis of the vitrector tube. The actuation mechanism may be or include a pneumatic, hydraulic, electromagnetic, or motor-driven mechanical actuation mechanism.

A fifth aspect of the invention relates to an injection device with integrated imaging capability. The injection device may include a hollow, tubular needle for piercing an injection site and delivering fluid to the site; an optical fiber disposed in parallel to the needle; a detector; a lens structure, mounted at a distal end of the needle and optically coupled to the optical fiber, for focusing light coupled into the lens structure from the optical fiber at a focus beyond the distal end of the needle; and an imaging engine for processing a signal received from the detector via the optical fiber and the lens structure so as to generate an image of a region about the focus. The lens structure may be, for example, a gradient-index lens. In one implementation, the gradient-index lens is integrated with the optical fiber and the diameter of the gradient-index lens is substantially equal to the diameter of the optical fiber. The lens structure may include an angle-cut lens coupled to an inner tube that has a diameter smaller than that of the tubular needle; in such embodiments, the inner tube may be coupled to the angle-cut lens and rotatably disposed within the needle. In certain embodiments, the lens structure is shaped so as to deflect the light and focus it off-axis. The device may further include an actuation mechanism for rotating and/or reciprocating the lens structure.

The optical fiber and the lens structure may be disposed inside and co-axially with the needle. In some embodiments, the inner diameter of the needle substantially matches the outer diameter of the lens structure and the needle includes a porous structure proximal to the lens structure for fluid egress from the needle. In alternative embodiments, the inner diameter of the needle is larger than the outer diameter of the lens structure for permitting fluid to flow around the lens structure and through an opening at the distal end of the needle. In yet another embodiment, the optical fiber and lens structure are disposed along an outer wall of the needle and adhered thereto. The lens structure may be recessed from the tip of the needle. The injection device may further include a forcing mechanism (e.g., a plunger, a source of pneumatic pressure, and/or a source of hydraulic pressure) for ejecting the fluid. In one implementation, the plunger includes a bore along its axis for accommodating the optical fiber.

A sixth aspect of the invention relates to an alternative injection device with integrated imaging capability. The device includes a hollow, tubular needle for piercing an injection site and delivering fluid thereto; an optical fiber coaxially disposed inside the needle; and a lens structure (e.g., an angle-cut lens and/or a gradient-index lens) optically coupled to the optical fiber and movable inside the needle from a position proximate the proximal end to a position proximate the distal end. The lens structure is configured such that, when positioned at the distal end, it focuses light coupled into the lens structure from the optical fiber beyond the distal end so as to image a region about the focus. The injection device may include a plunger for ejecting the fluid and pushing the lens structure towards the distal end.

A seventh aspect of the invention relates to a method of performing an injection and simultaneously imaging using an injection device comprising a hollow, tubular needle, an optical fiber in parallel with the needle, and a lens structure optically coupled to the optical fiber. In various embodiments, the method includes steps of piercing an injection site using the injection device; directing an optical beam, via the optical fiber and lens structure, to a region beyond the distal (ejection) end of the needle; and delivering a fluid to the region via the needle. The method may include pushing the lens structure towards the distal end. In one implementation, the method includes rotating the lens structure to scan a pattern. In certain embodiments, the method includes deflecting the optical beam to generate an off-axis focus using the lens structure. The off-axis focus may be scanned along an arc segment or an axis parallel to the axis of the tubular needle.

A eighth aspect of the invention relates to a surgical drill with integrated imaging capability. The surgical drill includes a drill bit with a bore along its axis; an optical fiber disposed in the bore; and, mounted in the bore at a distal end of the drill bit, a lens for focusing light coupled into the lens from the optical fiber to a focus beyond the distal end so as to image a region about the focus. The lens may be shaped so as to deflect the light and focus it off-axis, rotation of lens with the drill causing the focus to scan along a circular path. The surgical drill may further include a tube disposed in the bore and rotatable relative to the bore, and a second lens, mounted in the tube at a distal end thereof, for coupling the light from the optical fiber into the lens at the distal end of the drill bit. Both lenses may be angle-cut lenses that deflect the light, such that simultaneous rotation of the drill bit and the tube causes the focus to be moved along a scan pattern.

In an ninth aspect, the invention provides a system for endoscopically scanning a tissue sample. In various embodiments, the system includes a light source, a detector, an interferometer in optical communication with the light source and the detector, a handheld endoscopic imaging probe (in a sample arm of the interferometer) for communicating an optical beam from the light source to the sample, a memory, a user-operable activator (e.g., a footswitch, a button on the handheld endoscopic imaging probe, or a voice activation system), and an imaging engine. The imaging engine is responsive to the detector and to the activator, and processes an interferometric signal received from the detector. In response to user actuation of the activator, the imaging engine causes capture of an image or a video of the sample and storage thereof in the memory. In one implementation, the imaging engine is further responsive to a user command entered via the activator for causing display of the captured image or video.

In various embodiments, the handheld endoscopic imaging probe includes one or more lens structures shaped so as to direct the beam to an off-axis focus, and an associated actuation mechanism for scanning the focus laterally along the sample. The lens structure(s) may include or consist of a prism, a gradient-index lens or an angle-cut lens. The actuation mechanism may cause rotation of the lens structure(s) around an axis thereof or reciprocation of the lens structure(s) along an axis thereof. The actuation mechanism may include or consist of a pneumatic, hydraulic, electromagnetic, or motor-driven mechanical actuation mechanism. In addition, the actuation mechanism may include a transmission reconfigurable to dynamically alter the speed and/or the direction of actuation.

A tenth aspect of the invention relates to a method of endoscopically scanning a tissue sample using an interferometer in optical communication with a light source and a detector and an imaging engine in communication with the detector. In some embodiments, the method includes the steps of directing an optical beam from the light source to the sample; scanning the optical beam along the sample; processing a signal associated with the scanning of the optical beam; capturing, in response to a user request, an image and/or a video of the sample; and storing the captured image and/or video for later viewing.

The method may include displaying the captured image and/or video. In one implementation, the method further includes repeatedly performing the steps of capturing and displaying the image and/or video of the sample. Additionally, the method may include using a footswitch, a button, or a voice activation system to initiate the step of capturing. In some embodiments, the method includes deflecting the optical beam to generate an off-axis focus and scanning the off-axis focus laterally along the sample.

Where, in the above description of aspects of the invention, various features of embodiments are mentioned with respect to one aspect, such features may also be applicable to and used in one or more other aspects, as will be readily appreciated by a person of skill in the art from the summary and the following detailed description of the invention.

### BRIEF DESCRIPTION OF THE DRAWINGS

The foregoing will be more readily understood from the following detailed description of the invention, in particular, when taken in conjunction with the drawings, in which:

FIG. 1 is a cut-away view of an eyeball, illustrating typical incision points and insertion directions for instruments used in retinal surgery;

FIG. 2 is a schematic drawing of an optical tomography imaging system;

FIGS. 3A and 3B are schematic drawings of imaging probes in accordance with various embodiments, illustrating on-axis and off-axis focusing, respectively;

FIGS. 4A-4C are schematic drawings of imaging probes with various lens structures shaped so as to focus light off-axis in accordance with various embodiments;

FIG. 5 is a schematic drawing of an arc-shaped scan path in accordance with various embodiments, illustrating the deviation of the arc from a straight-line scan;

FIGS. 6A-6D are schematic drawings of handheld imaging probes in accordance with various embodiments, illustrating different mechanisms for actuation of the lens assemblies of the probes;

FIG. 7A is a schematic drawing of a dual-lens imaging probe with a rotary joint in accordance with one embodiment, and FIG. 7B is a schematic drawing of a rotary joint in accordance with another embodiment;

FIGS. 8A and 8B are schematic drawings of vitrector devices with rotating and reciprocating cutters, respectively, into which imaging probes are integrated in accordance with various embodiments;

FIGS. 9A-9C are schematic drawings of fluid-injection devices with integrated imaging probes in accordance with various embodiments;

FIGS. 10A-10C are schematic drawings of a fluid-injection device with integrated imaging probe in accordance with another embodiment, illustrating various positions of the imaging lens corresponding to various stages of plunger movement in the device; and

FIGS. 11A and 11B are schematic drawings of surgical drills with integrated imaging probes in accordance with various embodiments.

The shadings in the drawings are generally used for illustrative convenience, and not intended to denote any particular material.

### DETAILED DESCRIPTION

### 1. Systems and Methods for Endoscopic Imaging

### 1.1. OCT Imaging Systems

Imaging probes in accordance with various embodiments may be used with a variety of imaging modalities, including, for example, optical coherence tomography (OCT), confocal microscopy, fluorescence imaging, two-photon fluorescence imaging, Raman imaging, and coherent anti-Stokes Raman spectroscopy. Imaging systems that support these modalities are well-known to those of ordinary skill in the art of imaging (in particular, medical imaging). To provide just one example, and illustrate where the probe fits within the larger imaging system, an OCT system is described in the following. OCT imaging can provide one-, two-, or three-dimensional scans of biological tissues at sub-micrometer axial and/or lateral resolution. The advantages of OCT include high imaging resolution, real-time imaging, non-invasiveness, and compact size.

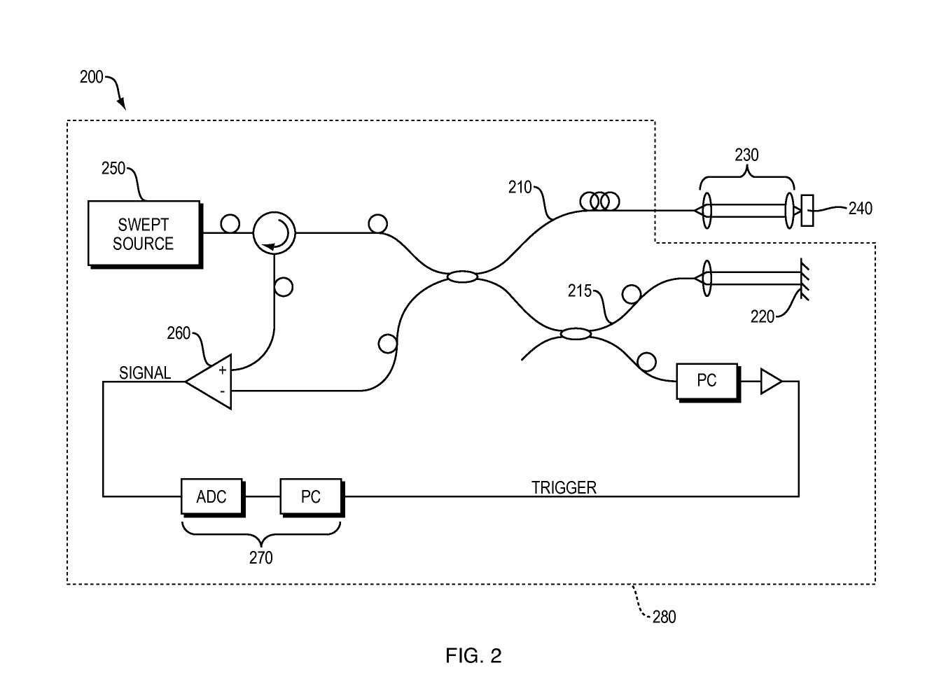

FIG. 2 depicts an exemplary OCT interferometry system 200 in accordance with embodiments of the present invention, but alternative systems with similar functionality are also within the scope of the invention. As depicted, OCT system 200 includes an interferometer with a sample arm 210 and a reference arm 215 including a reflector 220, optical components 230 for illuminating and collecting light from a sample of interest 240 in the sample arm, a light source 250, a photodetector 260, and an imaging engine 270 for data acquisition and processing. Light from light source 250 (which may be, e.g., a swept-source or tunable laser) travels through optical fibers of the sample and reference arms. Via the sample arm, the light illuminates or is focused onto the sample 240, which may include or consist essentially of, e.g., biological tissue. In a typical medical imaging application, the optical components 230 in the sample arm are the only components of the OCT system 200 that require contact with or close proximity to the area to be imaged (e.g., the eye). Accordingly, these components 230 may be provided in the form of a handheld imaging probe, which is, typically, operated by a surgeon or other physician performing medical diagnosis or treatment. The interferometer, light source 250, detector 260, and imaging engine 270 may be assembled in an imaging console 280 located remotely from the handheld probe.

Various features of sample 240 reflect the light in different amounts or from different depths. The reflected light is combined with light reflected by the reflector 220 (which typically includes or consists essentially of a mirror). Light reflected from features in the vicinity of the focus remains coherent, resulting in an interference pattern that provides information about the spatial dimensions and location of these features within sample 240. Light scattered off features that are located more than a coherence length away from the focus, on the other hand, are effectively filtered out by the interferometer 210. The interference pattern is captured with the photodetector 260, and processed by the imaging engine 270.

Imaging engine 270 may be a personal-computer-(PC)-based architecture, and may include a high-speed analog-to-digital converter (for example, on a PCI bus) that digitizes the output of photodetector 260 at a sampling rate ranging from several million samples per second to several billion samples per second. The digitized data may be processed by the PC processor based on straightforwardly implemented software instructions, e.g., instructions for performing a Fourier transform, processing the image signals and reconstructing images therefrom, and/or deriving biometrics or other quantitative data from the image data. Alternatively to using off-the-shelf-hardware such as a PC, the image acquisition and processing functionality may be implemented in dedicated hardware, such as an application-specific integrated circuit (ASIC), field-programmable gate array (FPGA), digital signal processor (DSP), graphical processing unit (GPU), or a combination of these devices. The imaging engine 270 may also have a customary user interface including, e.g., a monitor and/or input devices such as mouse and keyboard.

In various embodiments, the imaging engine 270 provides image reconstruction and display capabilities that enable real-time or near real-time viewing of the imaged target. For example, an LCD showing reconstructed OCT images may be mounted above a binocular microscope used by a retinal surgeon during a procedure. The surgeon can observe structures in the patient's eye (e.g., the retina) under the microscope as well as in OCT images on the display thereabove. While useful in many situations, such an arrangement may increase the risk and complexity of certain procedures that are highly sensitive to the exact position of the handheld imaging probe. For example, during a vitrectomy procedure, the surgeon may wish to image a region as close to the retina as possible (often less than one millimeter away). If the surgeon shifts focus from the view provided by the binocular microscope to the OCT display, he risks contacting and possibly damaging the patient's retina with the tip of the imaging probe. As another example, a surgeon, after having photocoagulated or ablated regions of the retina with a laser, may want to image the entire burn region to ensure that the results are satisfactory, requiring her to move the OCT probe across the area of interest. If the surgeon views the OCT image stream while scanning the probe, the probe tip may, inadvertently, contact the retina.

To avoid such problems, the imaging engine 270 includes, in various embodiments, means to capture snapshot images or videos for display over an extended period or at a later time, allowing the surgeon to image an area of interest, and then to remove the imaging probe from close proximity to the region of interest to safely view the captured image(s) immediately afterwards without risking damage to the area (e.g., the retina). For this purpose, the imaging engine 270 may include buffer memory (e.g., RAM) sufficient to store the captured image(s). In some embodiments, the surgeon may activate the capture-and-display modes by means of a footswitch or a button on the handheld probe (e.g., by pressing and holding the switch or button during capture mode and releasing it to display, or, alternatively, by pressing and releasing the button or switch once to activate the capture mode and a second time to activate the display mode). In alternative embodiments, switching between the two modes is achieved by voice activation. The images may, further, be stored long-term (e.g., in non-volatile memory) for review at a later time.

### 1.2. Single-Lens Imaging Probes

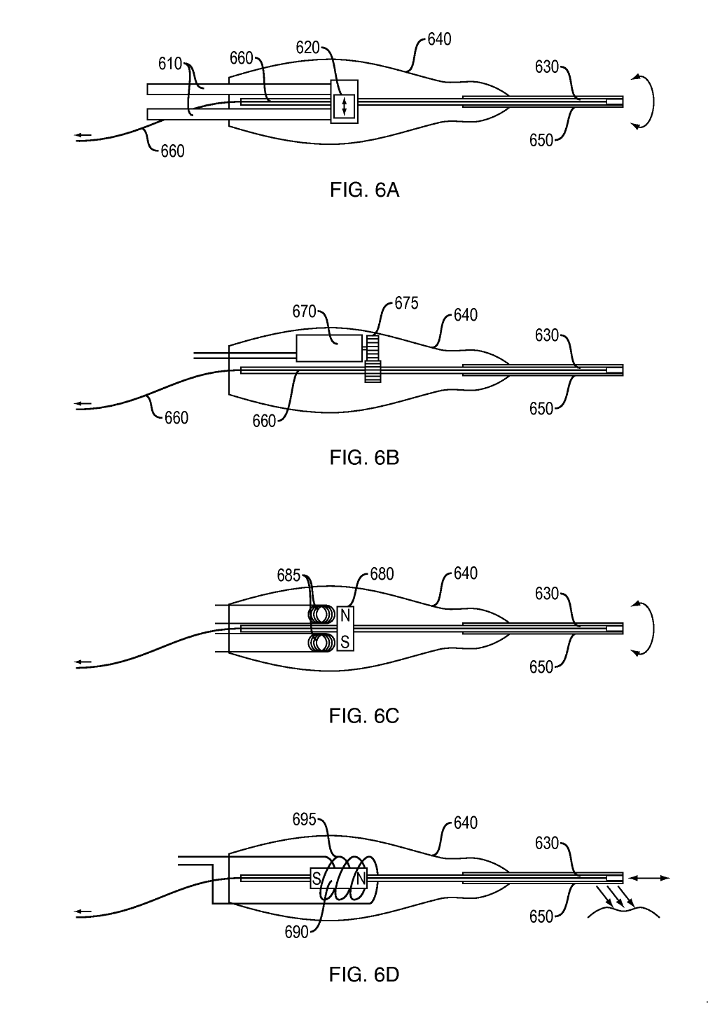

Various embodiments of the present invention are directed to imaging probes including (i) a hypodermic needle or similar tubing made of, e.g., stainless steel or a biocompatible polymer such as polyimide or polyether ether ketone, (ii) a lens structure including or consisting of, e.g., a gradient-index (GRIN) lens mounted in or on the needle at the distal end, and (iii) an optical fiber (e.g., a single-mode fiber) disposed inside the needle and optically coupled to the lens structure. Optionally, the imaging probe may further include (iv) an actuation mechanism for rotating or otherwise moving the tubing and lens structure (herein collectively referred to as the “lens assembly”), and, in some embodiments, (v) an outer tube that remains stationary when the lens assembly is moved so that it isolates the surrounding tissue from the movement, and which may (but need not) include a transparent window at the distal end to further isolate the tissue from the rotating lens structure. For use as a handheld probe, the various functional components listed above are typically enclosed in and/or attached to a suitably shaped and sized casing, as illustrated below with reference to FIGS. 6A-6D.

To facilitate miniaturization of the imaging probe for better access to body tissues, the outer diameter of the tubing and the diameters of the lens and optical fiber may be on a sub-millimeter scale. For example, if a commercial hypodermic needle is used for the tubing, a needle with a gauge of 20 or higher may be used. In general, the higher the gauge number, the smaller is the outer diameter of the needle. A 20-gauge needle, for example, has a nominal outer diameter of 908 μm; 23-gauge, 25-gauge, and 31-gauge needles have nominal outer diameters of 642 μm, 514 μm, and 260 μm, respectively. Of course, hypodermic needles used in imaging probes as described herein need not have outer diameters matching these nominal values, but may be customized needles having any diameter suitable for the particular application. In one embodiment, the lens and optical fiber both have a diameter of 125 μm. The fiber and lens may be housed, for example, in a regular 31-gauge needle, whose nominal inner diameter is 133 μm.

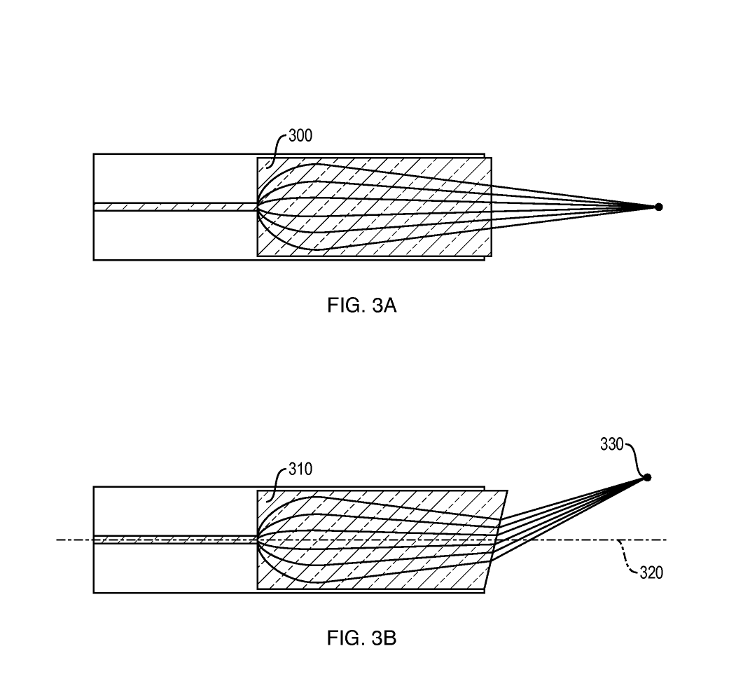

As conceptually illustrated in FIG. 3A, the lens structure 300 focuses light beyond the distal end at a desired imaging distance. In certain embodiments, depicted in FIG. 3B, the lens structure 310 is shaped so as to deflect the light beam away from the probe axis 320 (by up to) 90°, resulting in an off-axis focus 330. For example, the lens structure may be an angle-cut lens, as illustrated, or may include a prism or mirror surface at one end. The choice of lens structure depends on the application, and usually involves a cost/quality trade-off. An angle-cut lens generally causes a dispersion error (since different light rays are traveling slightly different distances in the lens medium), which is the greater, the larger the angle cut. Use of a prism typically results in superior imaging. However, an angle-cut lens is generally cheaper and easier to manufacture, which may render it preferable for single-use, disposable instruments.

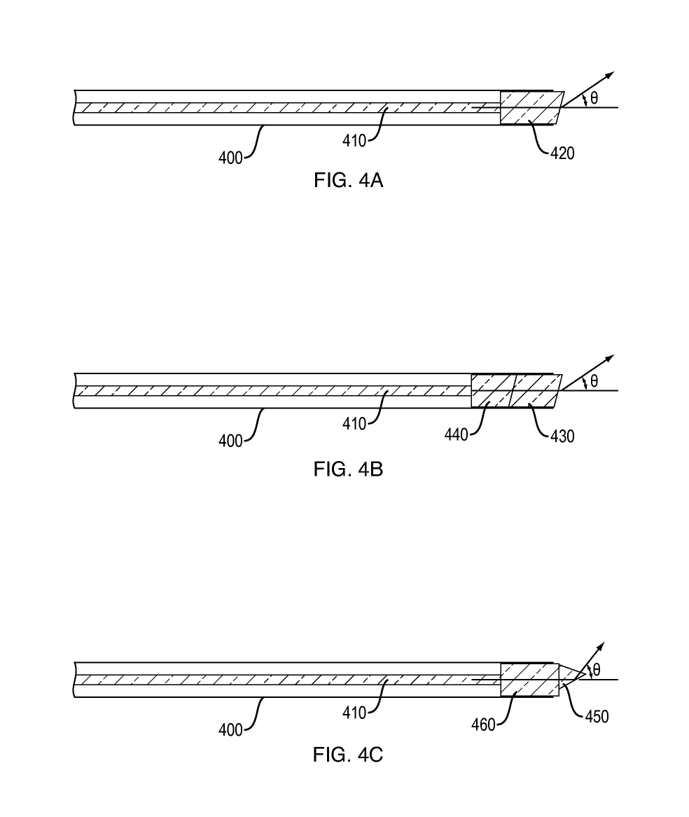

FIGS. 4A-4C illustrate various embodiments of imaging probes that focus light off-axis, each including tubing 400, an optical fiber 410, and a lens structure co-axially aligned with each other. In FIG. 4A, the lens structure consists of a polished, angle-cut focusing lens 420 directly attached (e.g., fusion-spliced, fiber-fused, or otherwise thermally bonded, or glued with optical-grade epoxy or another adhesive) to the optical fiber 410. In FIG. 4B, a polished focusing lens 430 that is angle-cut at both ends (as opposed to only at the distal end) is used, and is coupled to the optical fiber 410 via a fiber ferrule 440 (i.e., a plug, e.g., made of steel or glass, which holds the end of the fiber and aligns it against the lens). In FIG. 4C, the lens structure includes a prism 450 attached (e.g., thermally bonded or glued) to the focusing lens 460.

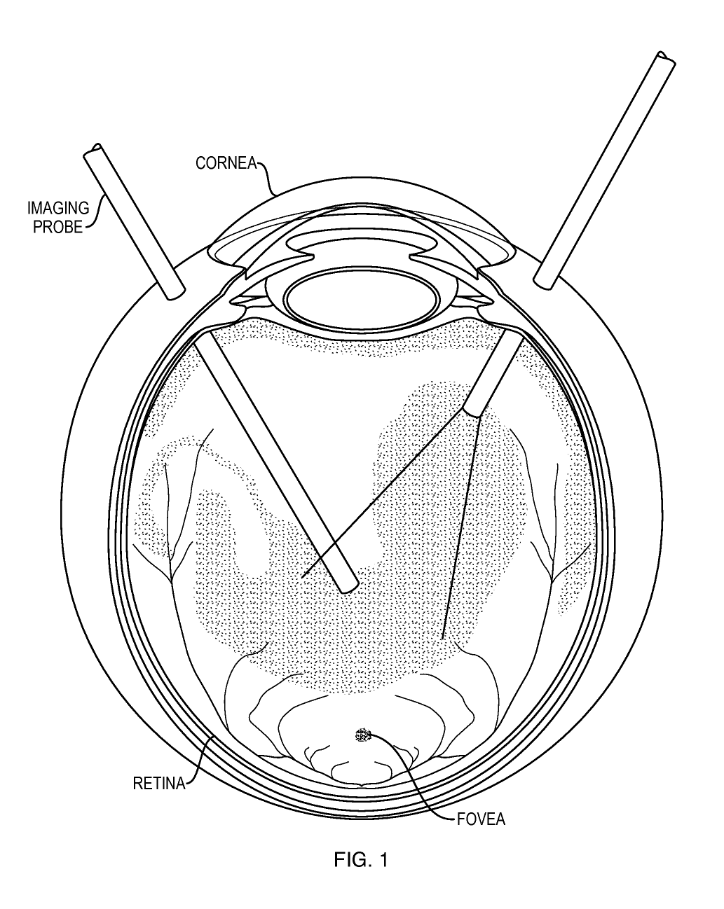

In use, the imaging probe is preferably positioned and oriented such that the focused beam is substantially perpendicular to the tissue surface to be imaged, e.g., such that the incidence angle, as measured between the beam and the surface normal, is less than 15°, preferably less than 5°. Advantageously, imaging probes that focus off-axis can achieve this perpendicularity, with suitably selected deflection angles, in anatomic environments that hinder introduction of the probe itself perpendicularly to the surface (as illustrated, e.g., in FIG. 1 for a retinal-surgery application). In retinal surgery, for example, imaging at an angle of deflection between about 30° and about 45° is desirable, rendering off-axis probes preferable over conventional forward-imaging or side-scanning probes. In addition, and perhaps more importantly, off-axis probes facilitate access to anatomic regions (such as, e.g., the central region of the retina around the fovea) that may be inaccessible, or accessible only with great difficulty, using an on-axis probe.

Moreover, off-axis probes may provide B- and C-scanning capabilities with a single lens (or lens structure). Specifically, in various embodiments, the lens assembly (i.e., the tubing and lens structure) is rotatable around or translatable along the probe axis, which facilitates lateral scanning of the beam, i.e., scanning across the tissue surface. In a single-lens probe that focuses on-axis, by contrast, rotation does not move the beam focus, and co-axial movement only shifts the focus in the direction of the beam. Therefore, existing forward-imaging scanning probes utilize at least two lenses. Herein, reference to a “single lens,” or a “single lens structure,” indicates that the beam-focusing and -deflecting optics at the distal end of the probe includes only one (i.e., no more than one) lens and, if applicable, an attached deflection component such as a prism. It does not necessarily imply that no additional lenses are used elsewhere in the imaging probe (although, in many embodiments, the focusing lens is the only lens in the probe).

When the lens assembly is rotated, the beam sweeps along a conical surface. A full rotation by 360° creates a circular scan pattern; a rotation by less than 360° results in an arc segment. The radius and curvature of the arc segment (and the circular scan pattern) can be determined through simple trigonometry, and depend on the angle of deflection (i.e., the angle between the off-axis light beam and the probe axis) and the distance to the target, which may be chosen and optimized for a particular application. Of course, when the beam rotates around the probe axis, it generally loses its original perpendicularity to the tissue surface (although curvature of the tissue surface may, in certain configurations, somewhat compensate for this effect). In a typical usage scenario, however, the lens assembly is rotated by significantly less than 360° (e.g., by an acute angle)) in one direction, and then by the same amount in the opposite direction (whereby the beam is returned to its original position), which limits the deviation from perpendicular incidence. In various embodiments, the rotation angle is in the range from 15° to 60°; e.g., it may be about 30°. Further, the probe may be oriented such that perpendicular incidence is achieved about mid-way along the scan path, which reduces the deviation from perpendicularity to about one half. (Also, non-perpendicular incidence is generally less important when the probe tip is closer to the tissue to be imaged.) Limiting the rotation of the fiber may also serve to minimize the strain on the fiber, reducing or eliminating the risk of damage to the fiber as well as undesirable effects of strain on, for example, the polarization of the light in the fiber.

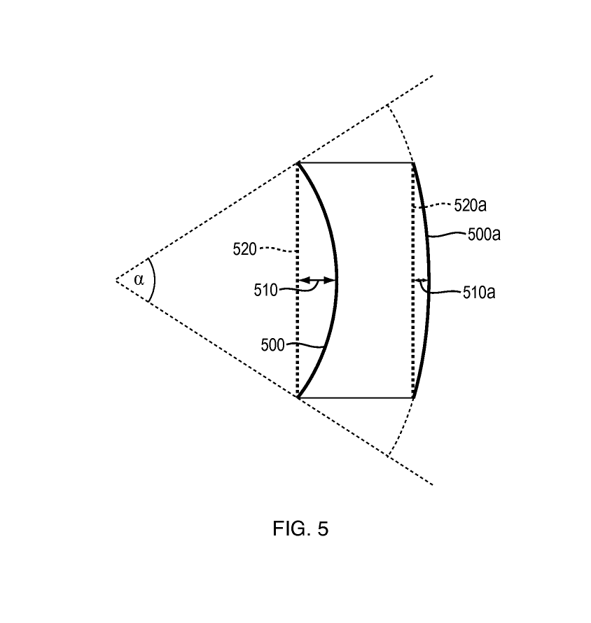

Off-axis rotating scanning probes are useful in many scenarios because they allow capturing a B-scan without requiring a full rotation of the lens assembly, which would necessitate a costly and complicated fiber-optic rotary joint, as used with most existing PARS probes. As illustrated in FIG. 5, the arc segment 500 resulting from partial rotation can—with some degree of error 510—be approximated as a straight line 520 (i.e., a “linear” scan). The approximation error 510 can be diminished by reducing the rotation angle α, which, however, also reduces the length of the arc segment. Alternatively or additionally, the approximation error for a given length of the segment may be reduced by increasing the distance to the target and/or the off-axis angle, thereby increasing the diameter of the circular scan pattern, as illustrated by a second set of arc segment 500a, straight line 520a, and approximation error 510a. With a properly designed imaging probe, the approximation error is in many applications small in comparison to the hand tremor and other unintentional and/or intentional movements of the operator holding the handheld probe; thus, it may be practically insignificant in these scenarios.

As each partial rotation of the lens assembly provides a separate B-scan, rotating the lens first clockwise and then by the same amount counterclockwise yields two B-scans. The imaging engine can utilize one or both of these B-scans; for example, it may display one B-scan image and discard the other, display two sequential B-scan images, or implement an averaging algorithm to combine the two B-scans to produce a single B-scan with an increased signal-to-noise ratio. The actuation of the lens assembly may be performed at a high speed, allowing for a high image-acquisition rate; for example, video-frame rates of about 25 frames per second may be supported.

In some embodiments, the lens assembly is translated forward and backward rather than rotated. For example, an off-axis probe that deflects the light beam by 45° or 90° can provide angled-scanning or side-scanning B-scan capabilities on an axis parallel with the lens assembly tubing, which may be useful for some applications. For example, in anterior eye surgery, it may be beneficial to insert the instrument in parallel to the structure of interest rather than pointing at it. Further, an imaging probe allowing B-scanning parallel to the probe axis is more suitable for incorporation into guillotine-type vitrectors, as described in detail below.

Several mechanical actuation mechanisms may be employed in scanning imaging probes to achieve the desired rotation or axial translation of the lens assembly. For example, FIG. 6A illustrates a pneumatically driven handheld imaging probe 600. An external pump and controller (not shown) may provide pneumatic power to the imaging probe 600 via two flexible air tubes 610. Pressure and vacuum are alternately applied to each tube 610 such that, when one tube is pushing, the other one is pulling. A piston 620 (or similar mechanism) connected to the tube of the lens assembly converts the push-pull forces to rotary forces, e.g., by means of a geared-rack and pinion-style configuration. This configuration is particularly useful in a vitreoretinal surgical setting, where surgical instruments such as, e.g., vitrectors, may be powered by the same push-pull mechanism as the imaging probe. Alternatively to a push-pull configuration, constant pressure or constant vacuum may also be used. For example, constant air flow may drive a turbine or propeller affixed to the needle, thereby causing the needle to rotate. Similar configurations can also be achieved utilizing hydraulic power in lieu of pneumatic power. As shown in FIG. 6A, the actuation mechanism may be contained, in large part, in a handpiece enclosure 640 (e.g., an ergonomically shaped enclosure). The lens assembly tube 630 and stationary outer tube 650 extend from the distal end of the enclosure 640; whereas the air tubes 610 and optical fiber 660 exit the enclosure at the proximal end.

In an alternative embodiment, illustrated in FIG. 6B, mechanical actuation is provided by a motor 670 that can be driven in both directions (e.g., a DC, brushless, stepper, or servo motor), and an associated transmission 675 (e.g., a gear or series of gears, a belt-drive, or a friction-based transmission) for transferring the rotational energy from the motor to the lens assembly tube 630. Certain implementations allow for dynamic changes of the configuration during use (such as, e.g., removal of gears by means of a lever that moves them into or out of place) that vary the speed or alter the direction of rotation.

Yet another set of embodiments utilizes electromagnetic drive mechanisms to achieve the rotary or forward-backward mechanical actuation. For example, the probe shown in FIG. 6C includes a permanent magnet 680 fixedly attached to the lens assembly tube 630, and one or more (as illustrated, a pair of) electromagnetic coils 685 that are fed by an alternating current to alternately attract the north and south poles of the magnet 680, thereby causing the magnet 680, and with it the lens assembly, to rotate. FIG. 6D applies a similar mechanism to achieve reciprocating motion along the probe axis. Here, a permanent magnet 690 is oriented parallel to the lens assembly tube 630, and is surrounded by a solenoid 695 driven by an alternating current. The magnetic field of the solenoid 695 alternately attracts and repels the magnet 690, thereby causing reciprocating motion of the lens assembly. It should be understood that the actuation mechanisms described herein are exemplary embodiments, and are not intended to limit in any way the mechanisms that may be used in imaging probes in accordance herewith.

Scanning imaging probes with a single-lens focusing optic provide cost savings and simplify manufacture and assembly, compared with multiple-lens designs as used in conventional PARS imaging probes. However, arc-shaped scan patterns (approximating the desired straight-line scan) may also be accomplished using a PARS probe or similar dual-lens design (as described in detail in the next section). PARS probes generally include two nested lens assemblies (each including a lens structure mounted to a tube) that are rotatable independently of each other, and an optical fiber that couples the inner lens to the external imaging console. Methods of using such probes to generate off-axis scan patterns generally involve rotating only the outer lens assembly, and holding the inner lens assembly, and with it the optical fiber, stationary. Although PARS probes typically include rotary joints, such a joint is not necessary if the probe is used like a single-lens probe. Even if the inner lens assembly is rotated (instead of or in addition to the outer lens), a rotary joint is not needed as long as the rotation is limited, e.g., to an acute angle in each direction.

1.3. Multiple-Lens Imaging Probes with Rotary Joints

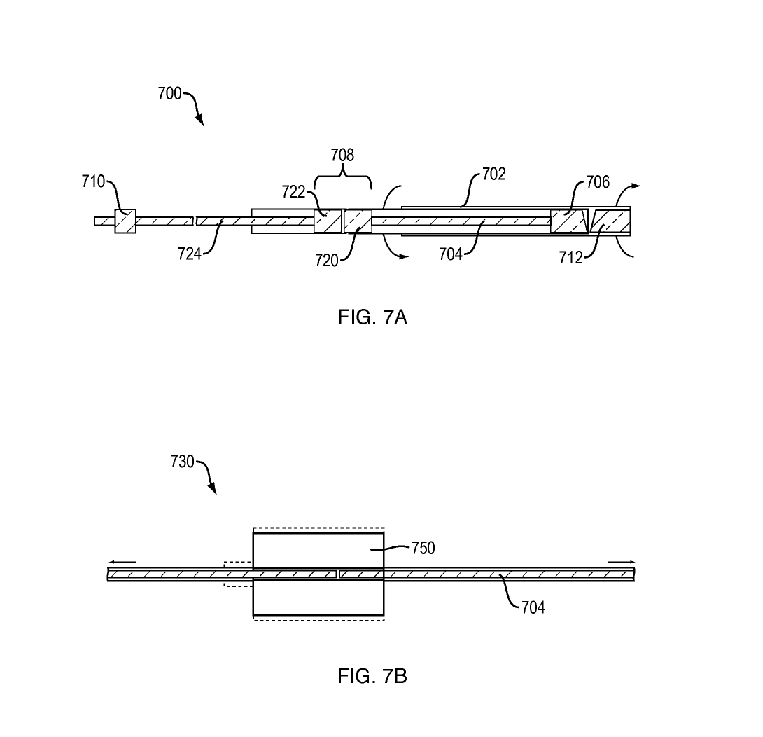

Various embodiments of the present invention are directed to scanning imaging probes (e.g., handheld probes for us in medical applications) that incorporate multiple rotating angle-cut lenses (or other deflecting lens structures) which collectively enable forward-scanning as well as laterally offset scanning (e.g., under a 45° or 90° angle with respect to the probe axis). The general configuration of the main functional components of such probes is illustrated in an exemplary embodiment shown in FIG. 7A. The imaging probe 700 includes two lens assemblies, each including an angle-cut lens mounted at the distal end of tubing such as, e.g., a hypodermic needle. One of the lens assemblies is “nested,” i.e., co-axially disposed, within the other one. The inner tube 702 contains an optical fiber 704, which couples the angle-cut lens 706 at the distal end of the tube via a fiber-optic rotary joint 708 to a fiber connector 710. The fiber connector 710 connects the imaging probe 700 to the imaging console (e.g., the console 280 depicted in FIG. 2). Light coupled from the fiber 704 into the lens 706 is deflected at the angled exit surface of the lens and, thereafter, at the entry and exit surfaces of the second lens 712. Depending on the relative orientations of the angled surfaces of the two lenses 706, 712, the light is focused on-axis or off-axis along a continuum of possible distances from the axis.

The inner tube 702 and the outer tube 714 are free to rotate independently of each other, thus allowing the relative lens orientations to be changed and the focus, as a result, to be moved along a scan pattern. An approximately linear (i.e., straight-line) scan pattern (i.e., in medical parlance, a typical B-scan) can be achieved by rotating the two lens assemblies at the same angular speed, but in opposite directions. A variety of other scan patterns (including, e.g., spirals and other patterns resembling Lissajous figures) can be achieved by varying the speed and/or direction of the two lens assemblies relative to each other. In typical usage, the lens assemblies may rotate a full 360° either clockwise or counter-clockwise, or they may rotate a lesser amount, e.g., 180°, in one direction and then rotate the same amount in the reverse direction. The first rotation scheme is more readily suited to a handheld probe powered by a standard motor, whereas the second rotation scheme is more readily suited to a pneumatic push-pull-driven probe (where the lens assembly is “pushed” in one rotary direction and then “pulled” in the opposite rotary direction).

Due to the rotation of the inner lens assembly, a means of providing rotation of the optical fiber 704 without interrupting or degrading the optical signal path is required. In standard dual-lens probes, a fiber-optic rotary joint is used for this purpose. However, currently available rotary joints are usually expensive, complex in design, and often engineered for long-term heavy use in extreme environments, which renders them unsuitable for disposable instruments, as are desired for some medical applications. Further, they are typically large, heavy, and bulky, and thus unsuited for incorporation into a handheld probe. To address these problems, the present invention provides, in various embodiments, simpler and smaller rotary joints that can be integrated into the handheld probe without negatively impacting signal quality.

FIG. 7A illustrates a rotary joint in accordance with one embodiment. The rotary joint is placed at a position proximal to the angle-cut lenses, and includes two cylindrically symmetric lenses 720, 722 (e.g., GRIN lenses) that are positioned co-axially inside the inner tube 702 and butt-coupled against each other. The lenses may be collimating lenses or, in some embodiments, converging lenses that focus the light slightly, still allowing the adjacent lens to capture all or most of the light. The small gap that typically remains between the two collimating lenses 720, 722 may be filled with an index-matching gel to avoid light scattering at the lens surfaces. The more distally located lens 720 is fused, or otherwise optically coupled, to the proximal end of the optical fiber 704 (which is, in turn, coupled to the angle-cut lens 706 of the inner lens assembly), and rotates with the inner lens assembly. The other lens 722 is coupled to the fiber connector 710 (typically, via a second optical fiber 724), and remains stationary. The collimating lenses 720, 722 may have fiber connectors (e.g., FC/APC) attached, which couple the lenses to the respective optical fibers 704, 724. Alternatively, they may include “pigtails” that can be fused or otherwise mated with the fiber ends.

An even simpler rotary joint 730 is shown in FIG. 7B. Here, the optical fiber 704 is directly coupled to a second optical fiber 740, which, in turn, connects the probe via the fiber connector 710 to the imaging console. The two fibers are positioned co-axially and held in place, e.g., in a fiber ferrule 750 (which may be made of glass or steel), such that axial misalignment is prevented. The ends of the two fibers are cleaved and/or polished, and butt-coupled against each other; the fiber ferrule (or similar arresting structure) prevents the ends from separating. The gap between the two fibers may be filled with index-matching gel to reduce reflections. In use, the fiber 704 connected to the angle-cut lens 706 at the distal end of the handheld probe is free to rotate while the fiber 740 connected to the imaging console is stationary. The rotary joint 730 illustrated in FIG. 7B is advantageous due to its structural simplicity and low cost, and may be preferable, for example, in disposable probes intended for single use. The rotary joint 708 shown in FIG. 7B, on the other hand, provides greater resilience to misalignment by incorporating collimating lenses 720, 722 at the interface between the two fibers 704, 724.

Regardless of its particular embodiment, the rotary joint (e.g., joint 708 or 730) is, where feasible, preferably located at a distance from the pair of angle-cut lenses that exceeds the coherence length of the light source. This way, reflections that may occur at the rotary-joint interface (e.g., the interface between the two fibers or between the two collimating lenses) are prevented from affecting the image quality. Currently available commercial light sources typically have coherence lengths in the range from about 4 mm to about 40 mm. While longer coherence lengths may be desirable for imaging within a longer range of depths, they may prevent placement of the rotary joint at a distance from the lens exceeding the coherence length (due to the limited length of the handheld probe). Thus, the selection of a light source with suitable coherence length generally involves a trade-off between image quality and the axial extent of the imaging region. In some embodiments, a light source with a coherence length in the range of 4-5 mm provides a sufficient scanning range in the axial direction, facilitating placement of the rotary joint a coherence length or more away from the lens.

In imaging probes with two or more lenses at the distal end, each lens assembly generally has its own associated actuation mechanism, although certain components of the mechanisms may be shared. Several methods of providing mechanical actuation to achieve the desired rotation are available. Some embodiments utilize one or multiple motors (e.g., DC, brushless, stepper, or servo motors), in conjunction with transmission means (such as one or more gears, a belt-drive, or a friction-based transmission) for transferring the energy from the motor to the lenses. The actuation mechanism(s) may also include a means of dynamically changing or altering the configuration during use (e.g., by including or removing gears by means of a lever that moves them into or out of place), for example, to provide variable speed or alter the direction of rotation.

Other embodiments utilize pneumatic power, e.g., are configured in a constant-pressure or constant-vacuum configuration or, alternately, in a push-pull configuration. For example, an external pump and controller may provide pneumatic power to the handheld probe (e.g., via one or multiple flexible tubes), which is used to turn one or multiple small turbines mounted coaxially to the lens assemblies, i.e., such that the tube of the lens assembly serves as the axis of the turbine and both rotate together when driven by the pneumatic pressure or vacuum. In one embodiment, a turbine can be mounted on both lens assembly tubes and configured such that the lens assemblies rotate in opposite directions. A similar configuration can also be achieved utilizing hydraulic power in lieu of pneumatic power. Additional embodiments incorporate drive mechanisms driven by a solenoid or other electromagnetic means, as described above with respect to FIG. 6C. In general, drive mechanisms for use in single-lens rotary probes are also applicable to multi-lens probes, and vice versa.

The features, structures and components described herein with respect to dual-lens imaging probes can be readily applied to probes with three or more lenses a well. A general multi-lens probe may, for example, include an arbitrary number of coaxial, nested lens assemblies, each comprising a tube and lens structure. The lens assemblies may be movable relative to one another, and any or all of them may have respective associated actuation mechanisms. One or more rotary joints as described above may be used to connect the probe, or the individual lens assemblies therein, to external equipment.

### 2. Integrated Tools

During surgical or other medical procedures, it is often desirable to image the region undergoing treatment to optimize the procedure, monitor treatment progress in the target tissue, and avoid unnecessary invasion into or damage of surrounding tissues. In the past, physicians were often limited to intermittent imaging, alternating with treatment, as anatomical and other constraints prevented the simultaneous use of endoscopic imaging probes and treatment devices. Advantageously, various imaging probes in accordance with the present invention are suitable, due at least in part to their compactness, for integration into various surgical and similar devices, facilitating imaging simultaneously with treatment. Accordingly, in certain embodiments, the invention provides endoscopic probes that combine imaging and treatment functionality. Specific embodiments are described below.

2.1. Vitrector with Integrated Imaging Probe

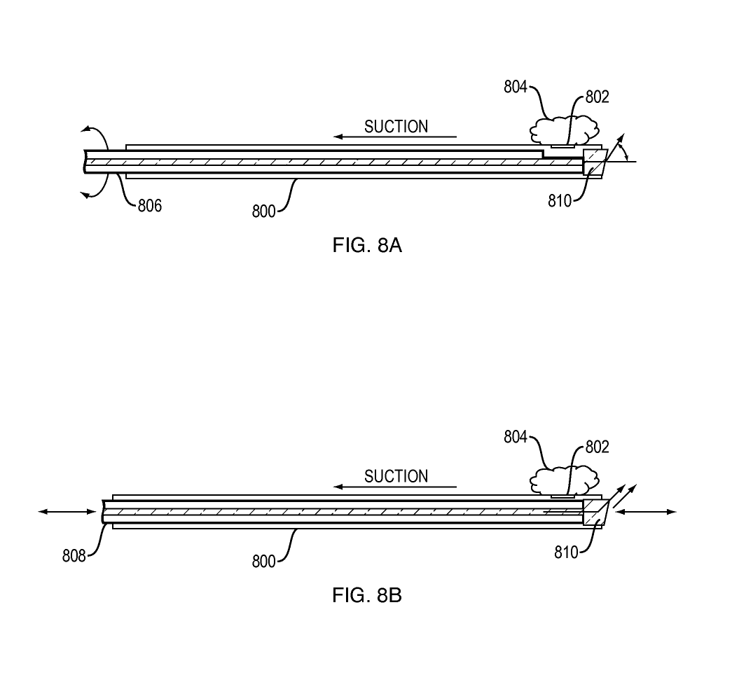

FIGS. 8A and 8B show embodiments of handheld imaging probes incorporated into vitrector devices. A vitrector is a surgical instrument used during vitrectomy—a procedure to remove vitreous from the vitreous body of the eye during a vitreoretinal surgery. The figures illustrate the operating principal of a vitrector. In general, vitrectors work by cutting through the vitreous and removing it through suction. More specifically, as shown in FIGS. 8A and 8B, the vitrector includes an outer tube 800 (e.g., hypodermic tubing) having a side port or window 802 at the distal end. Vitreous 804 admitted through this port 802 is drawn to the proximal end of the tube 800 via suction (as indicated by the arrow), and then removed. Inside the tube 800, a cutter tube (e.g., hypodermic tubing of a slightly smaller diameter) is coaxially disposed. The cutter tube moves relative to the outer tube 800, thereby creating shear forces at the port 802 that serve to cut the vitreous.

Vitrectors typically come in two general varieties: as rotating vitrectors or guillotine vitrectors. In rotating vitrectors, shown in FIG. 8A, a rotating cutter 806 rotates side-to-side across the port 802. In guillotine vitrectors, shown in FIG. 8B, a reciprocating cutter 808 moves forward and backward across the port 808. In either case, the shearing motion between the two tubings causes forces parallel to the port 802, which shear off any vitreous sucked into the space between the tubes. Vitrectors are commonly powered pneumatically. However, other actuation mechanisms (including those described for imaging probes with reference to FIGS. 6A-6D) may also be employed. For example, vitrectors may be electrically driven, using, e.g., a motor, solenoid, electromagnet, and/or other means.

To provide imaging functionality in the vitrector, a lens 810 may be attached to the cutter tube 806 or 808 at the distal end, and coupled to an optical fiber 812 that is run through the cutter tube along an axis thereof. The lens may be a simple forward-focusing lens that allows A-scans ahead of the probe. Such functionality is useful, for example, to detect the distance of the instrument tip to the retina and warn the surgeon (e.g., with an audio alarm) if it comes to close. Preferably, however, the lens 810 is angle-cut, as shown, or otherwise shaped to focus light off-axis. The lens 810 moves with the cutter tube, resulting in an arc-shaped scan pattern for a rotating cutter 806, and in a linear scan parallel to the probe axis for a reciprocating cutter 808. Thus, the same actuation mechanism that moves the cutter tube inherently also provides for the rotation or translation of the lens 810. This synergy between the surgical instrument and the imaging probe contributes to the compactness and small footprint of the combined device. In some embodiments, a non-moving transparent window is placed over the moving lens (e.g., mounted to the outer tube 800) to isolate the lens from the vitreous and thereby avoid spooling of the vitreous. (Note that the outer tube 800 itself is typically stationary and, thus, does not cause any spooling concerns.)

A vitrector with integrated imaging functionality is well-suited for vitreoretinal surgery, where surgeons have limited access to the surgical site in the eye, rendering multi-function devices that reduce or eliminate the need to swap out instruments highly desirable. OCT imaging is very useful both during and after a vitrectomy procedure for locating vitreous and ensuring that all excess vitreous has been removed, as unidentified vitreous can result in unintentional tears in the retina during a surgical procedure, complicating the procedure and diminishing patient outcome.

2.2. Injector with Integrated Imaging Probe

In various embodiments, a handheld imaging probe is integrated with a syringe for administering injections (e.g., of a drug, enzyme, or biochemical marker, and provided in the form of, e.g., a solution, emulsion, colloid, etc.). In some embodiments, simple A-scan imaging (e.g., OCT or two-photon fluorescence imaging) is facilitated, whereas other embodiments also provide B-scan capabilities.

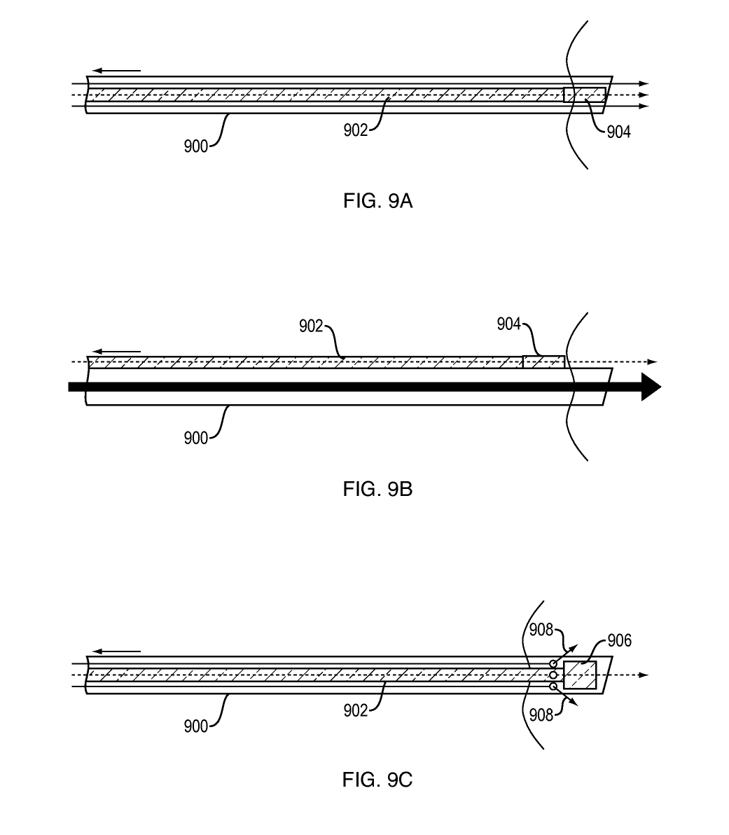

FIGS. 9A-9C illustrate various exemplary embodiments. In FIG. 9A, the device includes a hypodermic tube or needle 900 having an inner diameter sufficiently large to house an optical fiber 902 and lens 904 (e.g., GRIN lens), but allowing fluid flow (indicated with solid arrows) through the tube 900, around the fiber 902 and lens 904, to the site of injection. The specific sizing of the tube 902 and the lens 904 may be chosen depending on the intended site of the injection and the available access path to this site; lenses with diameters of 500 μm, 250 μm, and 125 μm are readily available, and smaller diameters can be achieved. As shown, the lens diameter may match that of the fiber 904. To expel fluid from the distal tip of the tube (which may be slanted or otherwise sharpened to facilitate penetration through the skin), a syringe plunger (not shown) may be pushed into a proximal end of the tube 900. In some embodiments, the distal end of the plunger halts at a distance from the distal end of the tube 900, and the optical fiber 902 is threaded through a hole in the side of the tube 900, located distal to the halting point, so as to not interfere with the plunger motion. In other embodiments, the plunger includes a bore along its axis that accommodates the optical fiber 902. Alternatively, instead of using a physical object for the plunger, pneumatic pressure may be used to perform the plunger function, i.e., to expel the fluid, without interfering with the fiber and lens.

In another embodiment, illustrated in FIG. 9B, the lens 904 and fiber 902 are disposed adjacent the injection tube 900. The lens 904 and fiber 902 may be secured to the outer surface of the tube 900 with a biocompatible epoxy or other adhesive, affixed by means of clamps or similar mechanical structures, held in place by a second tube that encloses both the injection tube 900 and the fiber 902 and lens 904, or installed in a separate tube welded to the side of the needle 900. The lens 904 may be recessed from the tip of the needle 900 such that, when the needle is inserted through the skin or other tissue surface at the site of injection, the lens 904 is adjacent to the surface.

In yet another embodiment, shown in FIG. 9C, a lens 906 of a diameter that substantially fills the inner diameter of the needle 900 is located at the distal end of the needle 900, and the injection fluid is expelled through slots, holes, ports, perforations, or another porous configuration 908 (e.g., produced by electrical discharge machining) in the needle wall proximal to the lens 906. In this embodiment, like in the one shown in FIG. 9A, the optical fiber 902 runs along the axis of the needle 900, and fluid flows around it.

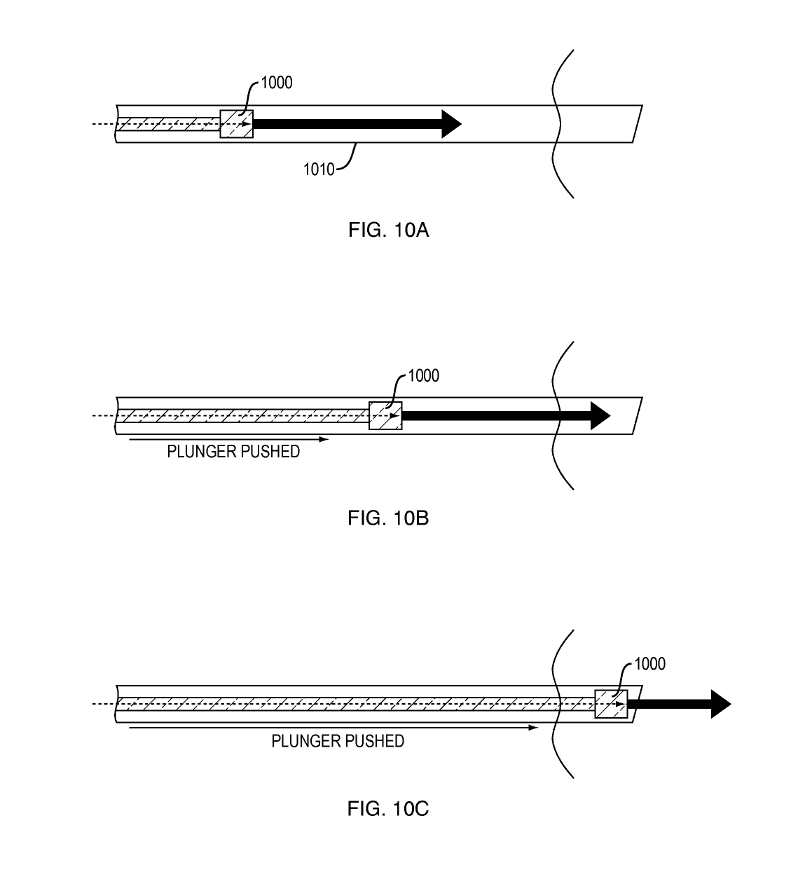

FIGS. 10A-10C illustrate an injection device, in three different stages, that provides for imaging only after the injection. Here, the lens 1000 is pushed through the needle core down the length of the injection needle 1010 as the syringe plunger (not shown) is pressed to cause fluid to be expelled from the distal end of the needle 1010. The lens 1000 ultimately stops at the tip of the needle 1010 to provide imaging at the injection site. In some implementations, the lens 1000 is attached to the front end of the plunger. In other implementations, it is simply pushed along with the fluid.

Integrating imaging capabilities into injection device is useful for imaging before, during, and/or after the injection, for example, to locate and target the optimal injection site, to observe the injection to verify that the desired injection site was properly targeted, to observe the effect of the injection, to monitor the physical response to the injection, etc. In many cases, this requires only A-scan capabilities. However, where B-scan capabilities are desired, they can be provided by straightforward modifications to the exemplary embodiments described above. For example, in the devices shown in FIGS. 9A and 9C, an inner tube of smaller diameter than the injection needle 900, disposed along the axis of the needle and surrounding the optical fiber 902, may be used, along with an angled lens, to form a lens assembly that can be rotated or translated to create a B-scan pattern, and which allows fluid to flow around it. As another example, in the embodiment of FIGS. 10A-10B, an angled lens may be attached to the plunger, and the plunger may be moved back and forth following injection to create a B-scan parallel to the needle axis. Additional embodiments providing B-scan capabilities may be implemented using mechanisms similar to those described in sections 1.2. and 1.3. above.

2.3. Surgical Drill with Integrated Imaging Probe

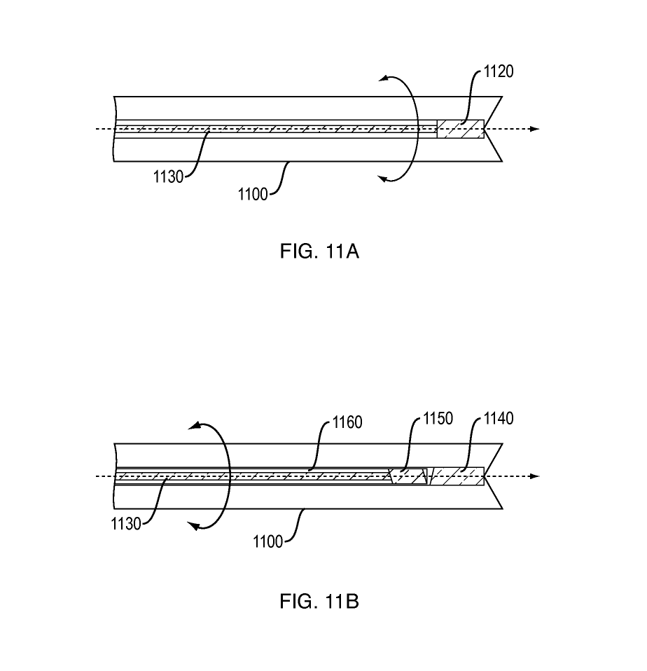

In some embodiments, an imaging probe is incorporated into a surgical drill device, as used, e.g., in orthopedic surgery. FIG. 11A, for example, shows a drill bit 1100 that contains, at a distal end a coaxial bore or channel 1110, a single lens 1120 providing A-scan capability. An optical fiber 1130 is threaded through the bore to couple the lens 1120, e.g., to an exterior imaging console. An imaging probe like this can be used, for example, to determine the distance from the tip of the drill bit 1100 to an interface (e.g., a bone/tendon interface). In the embodiment shown in FIG. 11A, the lens 1120 and fiber 1130 generally remain motionless while the drill bit rotates. However, if off-axis B-scan capability is desired, the lens 1120 may be replaced by an angle-cut lens that focuses light off axis and rotates along with the drill bit.

FIG. 11B shows a surgical drill with an integrated imaging probe that allows for B- and C-scans. The device includes two angle-cut lenses 1140, 1150. One lens 1140 is disposed inside the bore 1110 at the distal tip of the drill bit 1100 and rotates along with the drill bit 1100. The other lens 1150 is mounted inside a tube 1160 that is fitted into the bore 1110, and can rotate therein independently of the drill bit 1100. The second, interior lens 1150 is placed close to the lens at the distal tip, and is coupled to an optical fiber 1130 running through the interior tube 1160. If desired, the lens assembly formed by the tube 1160 and lens 1150 may be driven by the drill motor (or other actuation mechanism providing for the rotation of the drill bit). For example, a compound gear may be used to reverse the direction of rotation and thereby cause rotation of the two lenses at the same speed, but in opposite directions, resulting in an approximately linear scan pattern. Adjustments to the relative speed and direction between the two lenses may result in a variety of different scan patterns.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. For example, while various imaging probe embodiments are particularly suited for use in endoscopic devices, they may also be used in non-endoscopic applications. Further, the integration of imaging probes in accordance herewith into therapeutic devices is by no means confined to vitrectors, injection devices, and surgical drills, as were described in detail for illustrative purposes. Accordingly, the described embodiments are to be considered in all respects as only illustrative and not restrictive.

What is claimed is:

1. An injection device with integrated imaging capability, comprising:
a hollow, tubular needle for piercing an injection site and delivering fluid thereto;
an optical fiber disposed in parallel to the needle;
a detector;
mounted at a distal end of the needle and optically coupled to the optical fiber, a lens structure for focusing light coupled into the lens structure from the optical fiber at a focus beyond the distal end of the needle; and
an imaging engine for processing a signal received from the detector via the optical fiber and the lens structure so as to generate an image of a region about the focus.

2. The injection devices of claim 1, wherein the optical fiber and the lens structure are disposed co-axially inside the needle.

3. The injection device of claim 2, wherein the needle has an inner diameter substantially matching an outer diameter of the lens structure and comprises a porous structure proximal to the lens structure for fluid egress from the needle.

4. The injection device of claim 2, wherein the needle has an inner diameter larger than an outer diameter of the lens structure to permit fluid flow therearound to an opening at the distal end of the needle.

5. The injection device of claim 1, wherein the optical fiber and lens structure are disposed along an outer wall of the needle and adhered thereto.

6. The injection device of claim 5, wherein the lens structure is recessed from a tip of the needle.

7. The injection device of claim 1, wherein the lens structure is shaped so as to deflect the light and focus it off-axis.

8. The injection device of claim 1, further comprising an actuation mechanism for at least one of rotating or reciprocating the lens structure.

9. The injection device of claim 1, further comprising a forcing mechanism for ejecting the fluid.

10. The injection device of claim 9, wherein the forcing mechanism comprises at least one of a plunger, pneumatic pressure, or hydraulic pressure.

11. The injection device of claim 10, wherein the plunger comprises a bore along its axis for accommodating the optical fiber.

12. The injection device of claim 1, wherein the lens structure is a gradient-index lens.

13. The injection device of claim 12, wherein the gradient-index lens is integrated with the optical fiber, the gradient-index lens having a diameter substantially equal to a diameter of the optical fiber.

14. The injection device of claim 1, wherein the lens structure comprises an angle-cut lens coupled to an inner tube having a diameter smaller than a diameter of the tubular needle, the inner tube being coupled to the angle-cut lens and rotatably disposed within the tubular needle.

15. An injection device with integrated imaging capability, comprising:
a hollow, tubular needle for piercing an injection site and delivering fluid thereto;
an optical fiber coaxially disposed inside the needle; and
a lens structure, optically coupled to the optical fiber and movable inside the needle from a position proximate a proximal end thereof to a position proximate a distal end thereof,
wherein the lens structure, when positioned at the distal end, is configured to focus light coupled into the lens structure from the optical fiber beyond the distal end so as to image a region about the focus.

16. The injection device of claim 15, further comprising a plunger for ejecting the fluid and pushing the lens structure towards the distal end.

17. The injection device of claim 16, wherein the lens structure comprises an angle-cut lens.

18. The injection device of claim 15, wherein the lens structure comprises a gradient-index lens.

19. A method of performing an injection and simultaneously imaging using an injection device comprising a hollow, tubular needle, an optical fiber in parallel with the needle, and a lens structure optically coupled to the optical fiber, the method comprising steps of:
piercing an injection site using the injection device;
directing an optical beam, via the optical fiber and lens structure, to a region beyond the distal end of the needle; and
delivering a fluid to the region via the needle.

20. The method of claim 19, further comprising pushing the lens structure towards the distal end.

21. The method of claim 19, further comprising rotating the lens structure to scan a pattern.

22. The method of claim 19, further comprising deflecting the optical beam to generate an off-axis focus using the lens structure.

23. The method of claim 22, further comprising scanning the off-axis focus along an arc segment.

24. The method of claim 22, further comprising scanning the off-axis focus along an axis parallel to an axis of the tubular needle.
